(19) United States
(12) Patent Application Publication
(10) Pub. No.: US 2026/0201348 A1
(43) Pub. Date: Jul. 16, 2026

(54) CARBON ANTIOXIDANT ENZYME MIMIC, ANTI-INFLAMMATORY COMPOSITION COMPRISING THE SAME, AND METHOD FOR PREPARING THE SAME

(71) Applicant: INDUSTRY-UNIVERSITY COOPERATION FOUNDATION HANYANG UNIVERSITY ERICA CAMPUS, Ansan-si, Gyeonggi-do (KR)

(72) Inventors: Jong Ho KIM, Ansan-si (KR); Da Bin YIM, Ansan-si (KR); Chan Hee CHOI, Ansan-si (KR)

(21) Appl. No.: 19/562,074

(22) Filed: Mar. 10, 2026

Related U.S. Application Data
(63) Continuation of application No. PCT/KR2024/097162, filed on Dec. 19, 2024.

(30) Foreign Application Priority Data
Jan. 30, 2024 (KR) 10-2024-0014464
Jan. 30, 2024 (KR) 10-2024-0014468

(51) Int. Cl. C12N 9/08; A61K 38/00; A61P 19/02; A61P 29/00

(52) U.S. Cl. CPC C12N 9/0065; A61P 19/02; A61P 29/00; A61K 38/00

(57) ABSTRACT

A carbon antioxidant enzyme mimetic is provided, wherein a surface of the carbon antioxidant enzyme mimetic comprises a first functional group selected from a carbonyl group or an amine group and a second functional group comprising a hydroxyl group. The carbon nanoparticle-based antioxidant enzyme mimetic effectively scavenges reactive oxygen species and reactive nitrogen species without metal ions, and exhibits anti-inflammatory effects by reducing inflammatory cytokine levels and increasing expression of endogenous antioxidant enzymes.

### CROSS-REFERENCE TO RELATED APPLICATION

This application is a bypass continuation of pending PCT International Application No. PCT/KR2024/097162, which was filed on Dec. 19, 2024, and which claims priority to and the benefit of Korean Patent Application Nos. 10-2024-0014468 and 10-2024-0014464, each filed with the Korean Intellectual Property Office on Jan. 30, 2024, the disclosures of which are incorporated herein by reference in their entirety.

### TECHNICAL FIELD

The present invention relates to a carbon antioxidant enzyme mimetic and a method for preparing the same, and more particularly, to a novel carbon antioxidant enzyme mimetic that exhibits catalytic activity for scavenging reactive oxygen species (ROS) even in the absence of metal ions, and a method for preparing the same.

### BACKGROUND

Intracellular reactive oxygen species and reactive nitrogen species act as mediators of inflammatory responses, and excessive expression of reactive oxygen species and reactive nitrogen species induces excessive expression of pro-inflammatory cytokines, thereby causing various inflammatory diseases.

Pro-inflammatory cytokine antagonists used as conventional therapeutics for inflammatory diseases have mostly failed due to low therapeutic efficacy and severe side effects. Accordingly, antioxidant therapies that treat inflammatory diseases by scavenging reactive oxygen species and reactive nitrogen species have been developed. However, conventional antioxidants and antioxidant enzymes suffer from low intracellular activity and thus cannot effectively scavenge intracellular reactive oxygen species and reactive nitrogen species.

To address this issue, various nanomaterial-based antioxidant materials have been developed. However, most conventional nanomaterials are metal oxide-based nanomaterials, which induce cytotoxicity when administered at high concentrations, thereby limiting their applicability as antioxidants.

### SUMMARY OF THE INVENTION

### Technical Problem

Accordingly, an object of the present invention is to provide a mimetic capable of effectively scavenging reactive oxygen species and reactive nitrogen species.

### Technical Solution

To achieve the above object, the present invention provides a carbon antioxidant enzyme mimetic, wherein a surface of the carbon antioxidant enzyme mimetic comprises a first functional group selected from a carbonyl group or an amine group, and a second functional group comprising a hydroxyl group.

In one embodiment of the present invention, the first functional group and the second functional group simultaneously participate in an oxidation reaction of a reactive oxygen species, HOO⁻.

In one embodiment of the present invention, the carbon antioxidant enzyme mimetic selectively oxidizes or reduces the HOO⁻ species.

In one embodiment of the present invention, after the oxidation reaction, the carbonyl group, which is the first functional group, is converted into a hydroxyl group, and the hydroxyl group, which is the second functional group, subsequently binds again to the reactive oxygen species HOO⁻ to reduce the same.

In one embodiment of the present invention, the carbon antioxidant enzyme mimetic removes HOO⁻ according to the following reaction scheme:

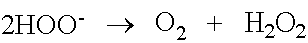

In one embodiment of the present invention, the carbon antioxidant enzyme mimetic may have crystallinity, and in this case, a crystalline domain size of the crystalline carbon antioxidant enzyme mimetic is 0.2 nm or greater.

The present invention further provides a method for preparing a carbon antioxidant enzyme mimetic, comprising a step of condensation polymerization of an organic compound having a first functional group selected from a carbonyl group or an amine group and a second functional group comprising a hydroxyl group, wherein particles obtained after the condensation polymerization have surfaces on which the first functional group and the second functional group are bonded.

In one embodiment of the present invention, the condensation polymerization step is a solvothermal reaction in which heat is applied to the organic compound in a solution, wherein the solvothermal reaction is performed by microwave irradiation, and crystallinity of the carbon antioxidant enzyme mimetic is determined according to the temperature and time of the solvothermal reaction.

In one embodiment of the present invention, the organic compound is caffeic acid or norepinephrine.

### Effects of the Invention

According to the present invention, a carbon nanoparticle-based antioxidant enzyme mimetic can effectively scavenge reactive oxygen species and reactive nitrogen species without the use of metal ions. In addition, the mimetic exhibits anti-inflammatory effects by reducing inflammatory cytokines and increasing the expression of endogenous antioxidant enzymes in cells.

### BRIEF DESCRIPTION OF THE IMAGES

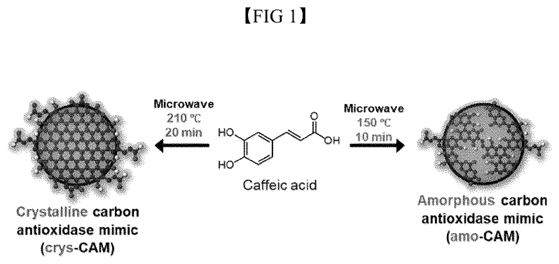

FIG. 1 is a schematic diagram illustrating the synthesis of a crystalline or amorphous carbon antioxidase mimics (crys-CAM or amo-CAM) through a spontaneous solvothermal reaction of caffeic acid.

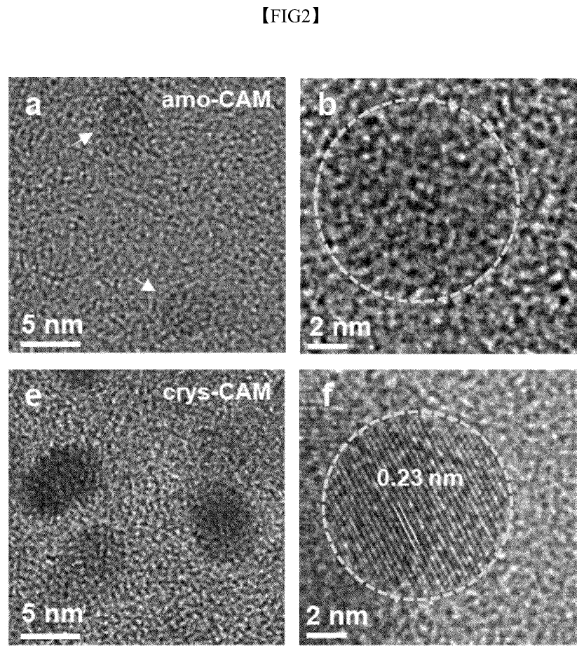

FIG. 2 shows transmission electron microscopy (TEM) images of crystalline or amorphous carbon-based antioxidant enzyme mimetics prepared according to the present invention, wherein the upper image corresponds to the amorphous structure and the lower image corresponds to the crystalline structure.

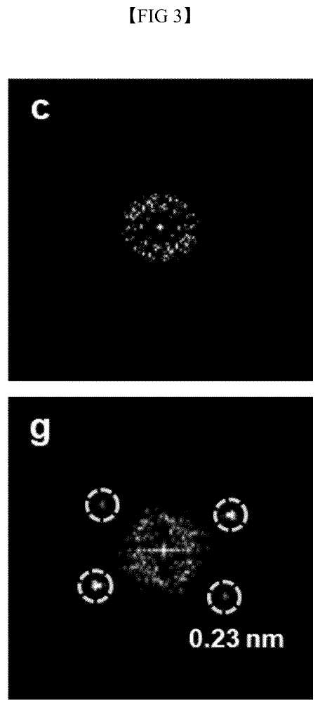

FIG. 3 shows Fast Fourier Transform (FFT) patterns of crystalline or amorphous carbon-based antioxidant enzyme mimetics prepared according to the present invention, wherein the upper image (c) corresponds to the amorphous structure and the lower image (g) corresponds to the crystalline structure.

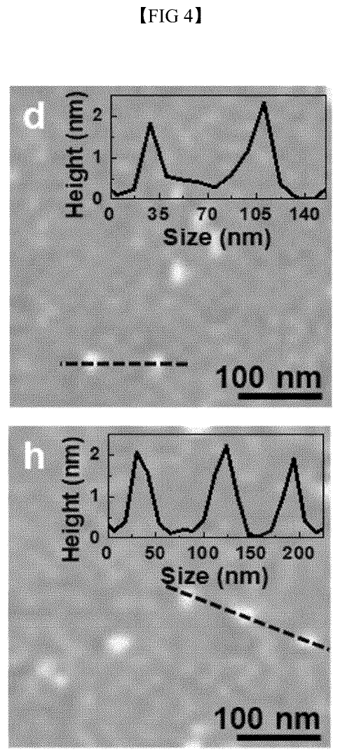

FIG. 4 shows atomic force microscopy (AFM) images of crystalline or amorphous carbon-based antioxidant enzyme mimetics prepared according to the present invention, wherein the upper image (d) corresponds to the amorphous structure and the lower image (h) corresponds to the crystalline structure.

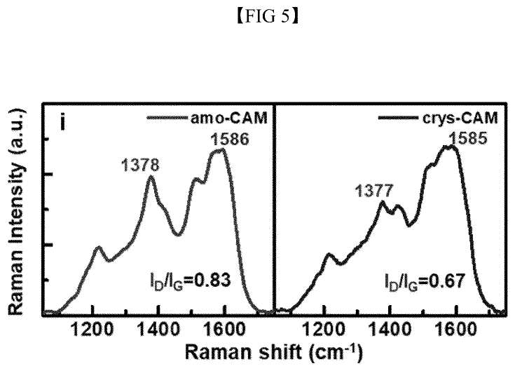

FIG. 5 shows ultraviolet-visible (UV-Vis) absorption spectra of crystalline or amorphous carbon-based antioxidant enzyme mimetics prepared according to the present invention.

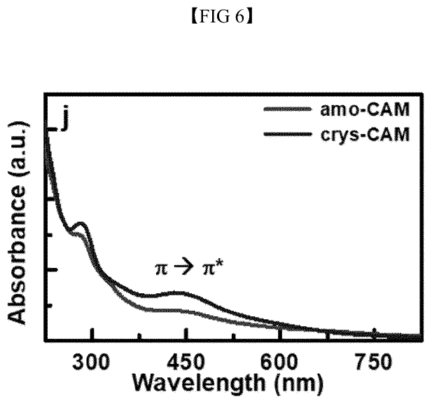

FIG. 6 shows Raman spectrum of crystalline or amorphous carbon-based antioxidant enzyme mimetics prepared according to the present invention.

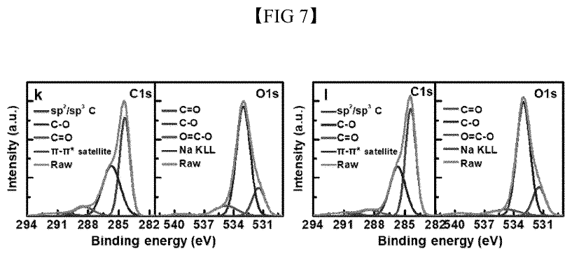

FIG. 7 shows X-ray photoelectron spectroscopy (XPS) spectra of crystalline or amorphous carbon-based antioxidant enzyme mimetics prepared according to the present invention, wherein the left graph (k) corresponds to the amorphous structure and the right graph (i) corresponds to the crystalline structure.

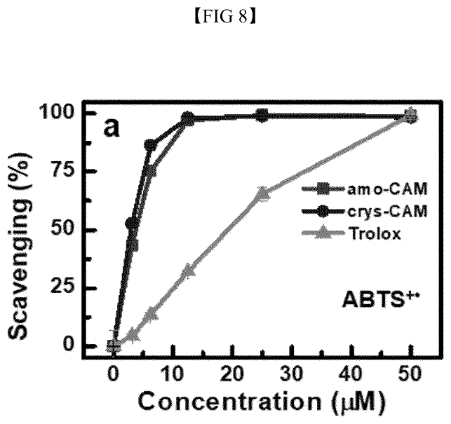

FIG. 8 shows scavenging activity of crys-CAM, amo-CAM, and Trolox against ABTS radicals according to Example 1 of the present invention, and an analysis of the antioxidant mechanism.

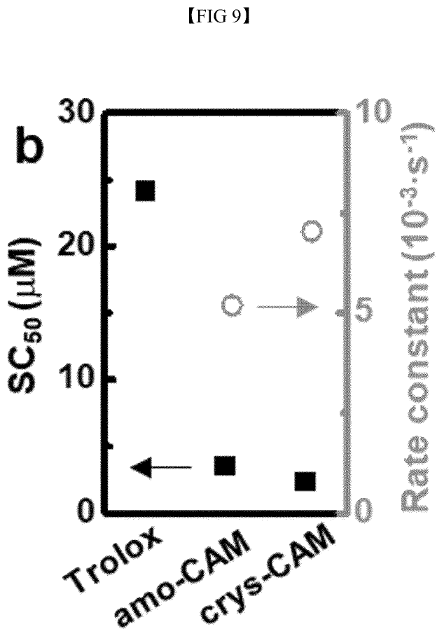

FIG. 9 shows concentrations required to scavenge 50% (SC₅₀) of ABTS radicals and scavenging rate constants against extracellular peroxide for crys-CAM and amo-CAM according to Example 1.

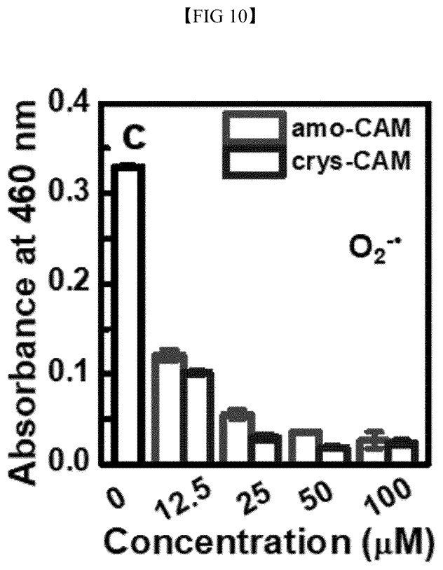

FIG. 10 shows concentration-dependent scavenging activity of crys-CAM and amo-CAM against extracellular superoxide according to Example 1.

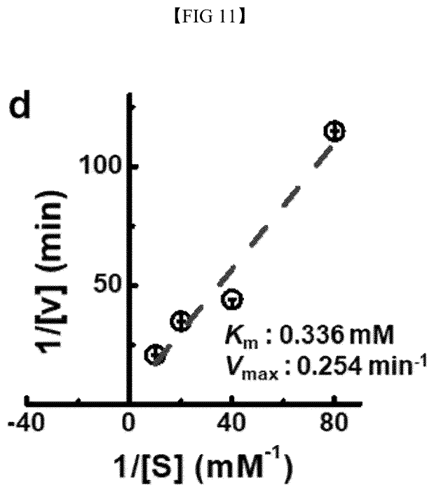

FIG. 11 shows a Lineweaver-Burk plot for superoxide scavenging by SOD-like crys-CAM according to Example 1.

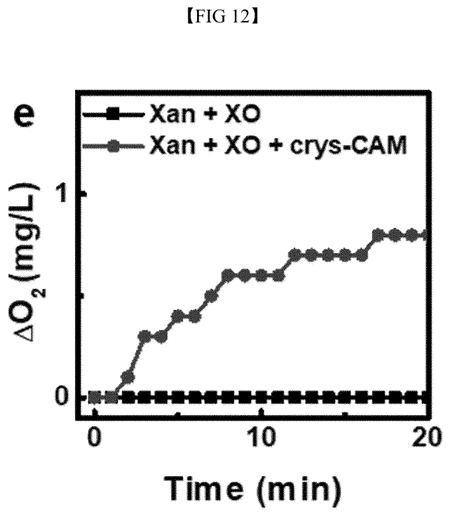

FIG. 12 shows analysis results of molecular oxygen converted from superoxide by SOD-like crys-CAM according to Example 1.

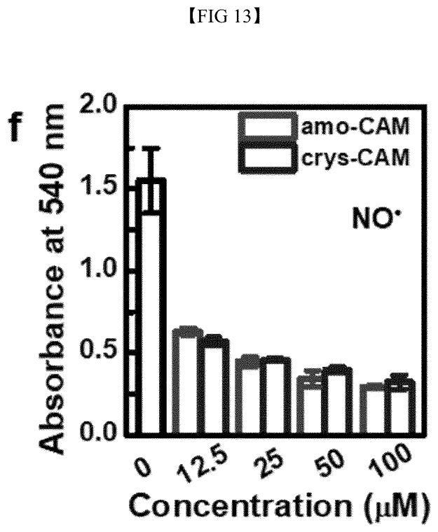

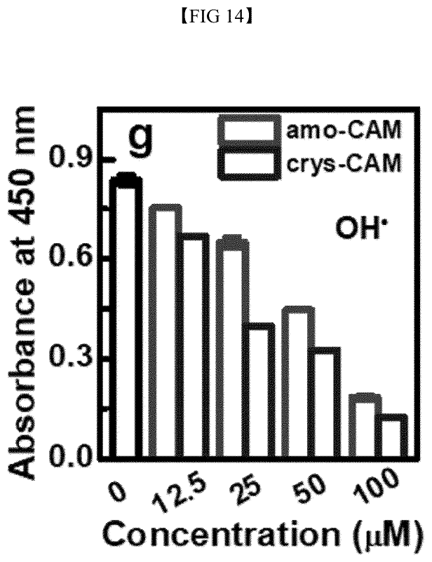

FIG. 13 shows scavenging activity of crys-CAM and amo-CAM against extracellular nitric oxide, and FIG. 14 shows scavenging activity against hydroxyl radicals.

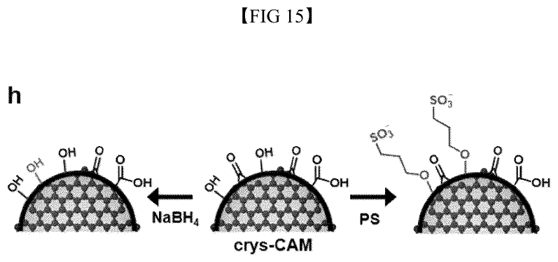

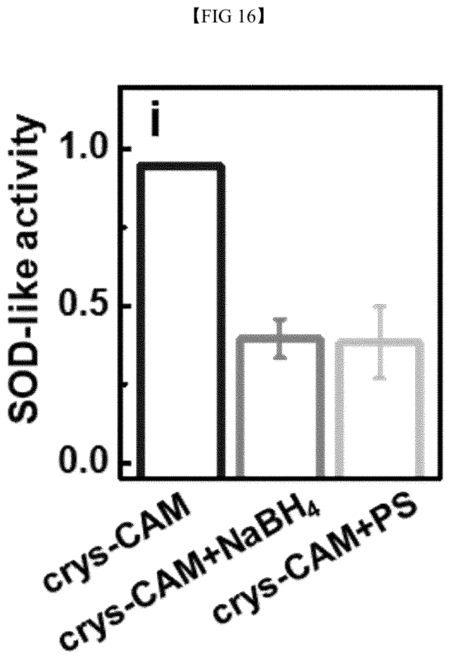

FIG. 15 is a schematic diagram illustrating treatment of crys-CAM of Example 1 with NaBH₄ and 1,3-propane sultone (PS), and FIG. 16 shows SOD-like activity of untreated crys-CAM and crys-CAM of Example 1 treated with NaBH₄ and 1,3-propane sultone (PS).

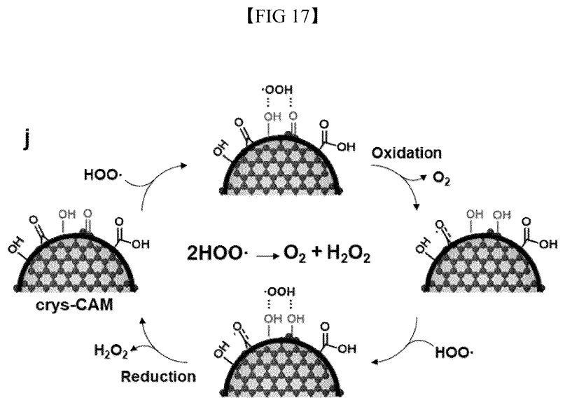

FIG. 17 illustrates the mechanism of SOD-like activity of crys-CAM according to Example 1.

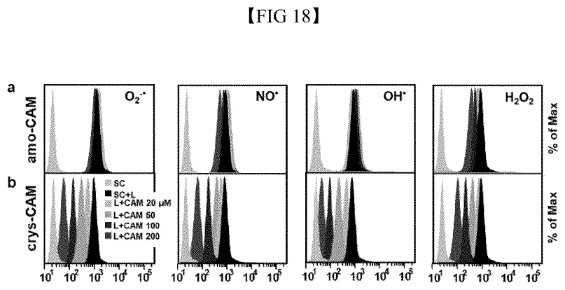

FIG. 18 compares scavenging abilities against intracellular superoxide, nitric oxide, hydroxyl radicals, and hydrogen peroxide in cells treated with amo-CAM (a) and crys-CAM (b).

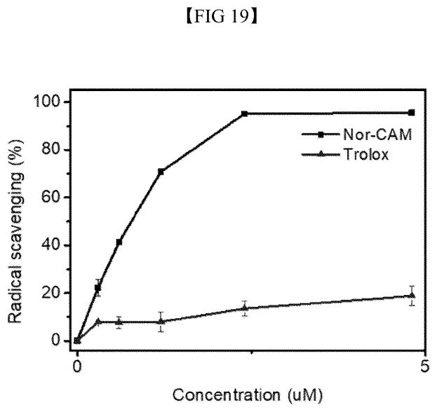

Referring to FIG. 19, it is confirmed that Example 2 having an amine group exhibits antioxidant properties identical or similar to those of Example 1.

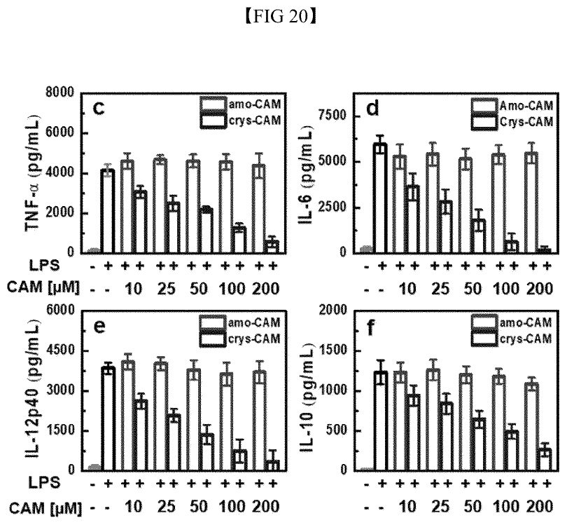

FIG. 20 shows secretion levels of TNF-α (acute inflammation), IL-6 (chronic inflammation), IL-12p40 (chronic inflammation), and IL-10 (anti-inflammatory) in cells treated with amo-CAM (a) and crys-CAM (b).

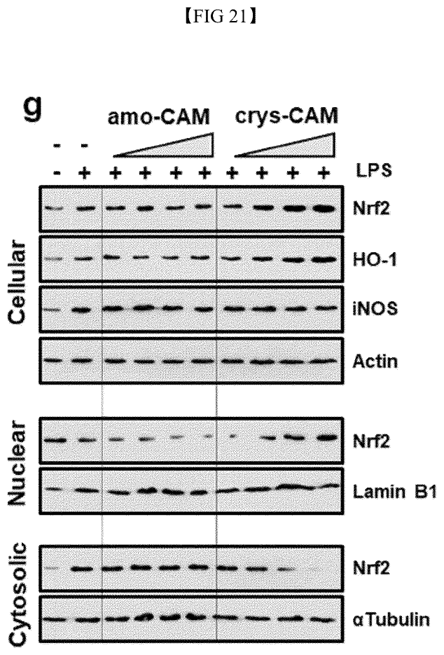

FIG. 21 shows Western blot analysis of the Nrf2 signaling pathway in LPS-stimulated Bone Marrow-Derived Macrophage (BMDM) after treatment with amo-CAM or crys-CAM.

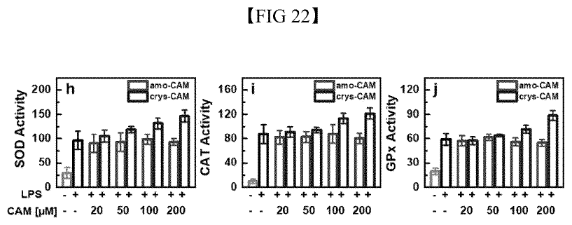

FIG. 22 shows intracellular activities of Nrf2-related innate antioxidant enzymes, including SOD, CAT (i), and GPx (j), in LPS-stimulated BMDM.

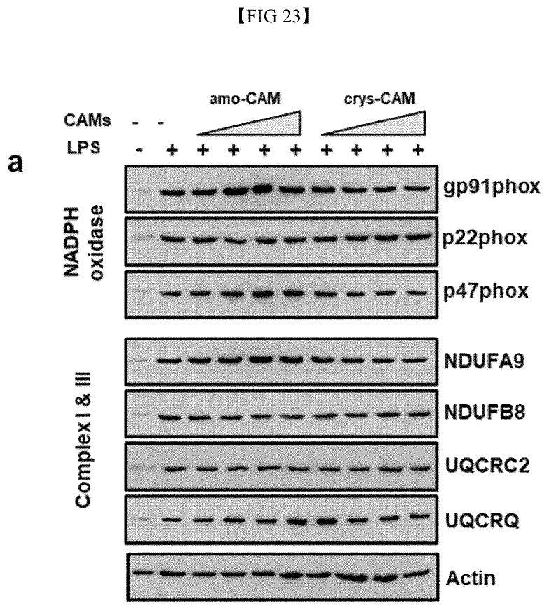

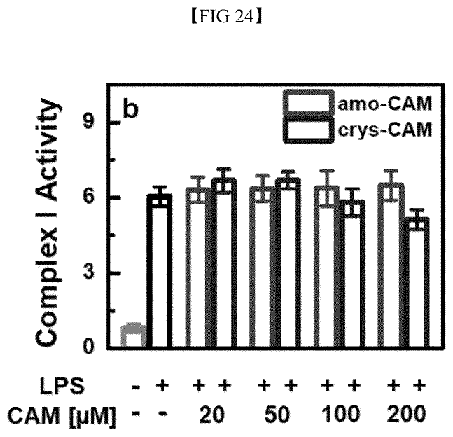

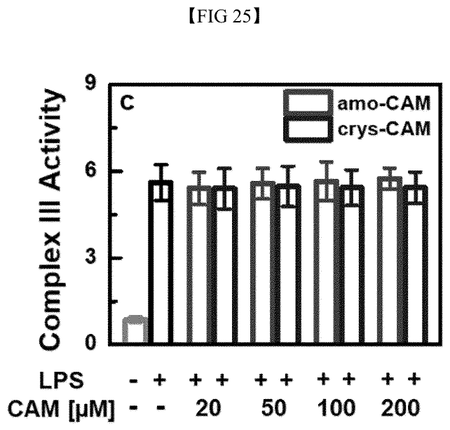

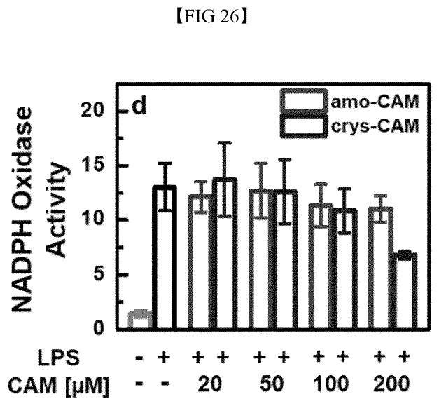

FIGS. 23-26 show Western blot analysis of mitochondrial ROS-generating proteins and activities of Complex I, Complex III, and NADPH oxidase in LPS-stimulated BMDM treated with amo-CAM or crys-CAM.

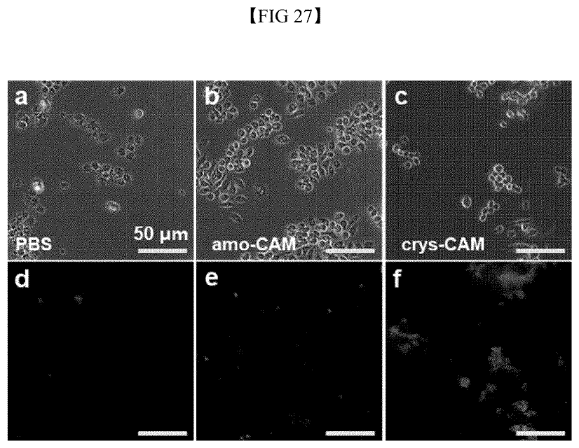

FIG. 27 shows optical and fluorescence images of RAW264.7 cells treated with PBS, amo-CAM, or crys-CAM, illustrating intracellular uptake pathways and immune enhancement.

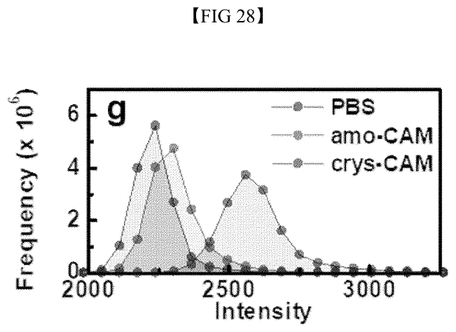

FIG. 28 quantifies fluorescence intensity derived from FIG. 27.

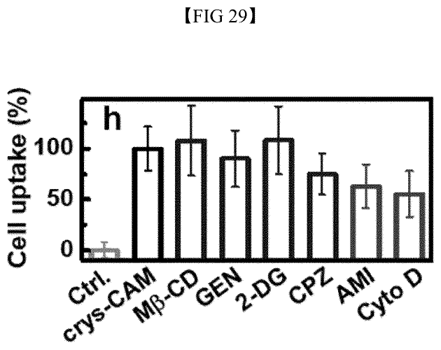

FIG. 29 shows effects of endocytosis inhibitors on cellular uptake of crys-CAM in RAW264.7 cells.

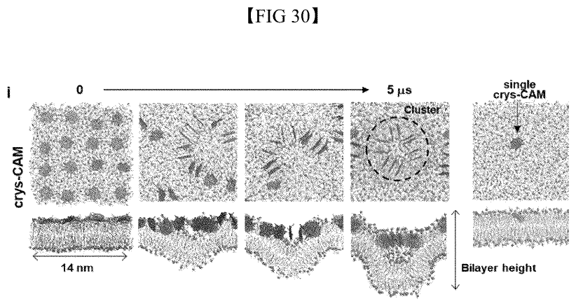

FIG. 30 shows snapshots of top and side views of cell membranes adsorbing crys-CAM (red beads) or amo-CAM (blue beads) over time.

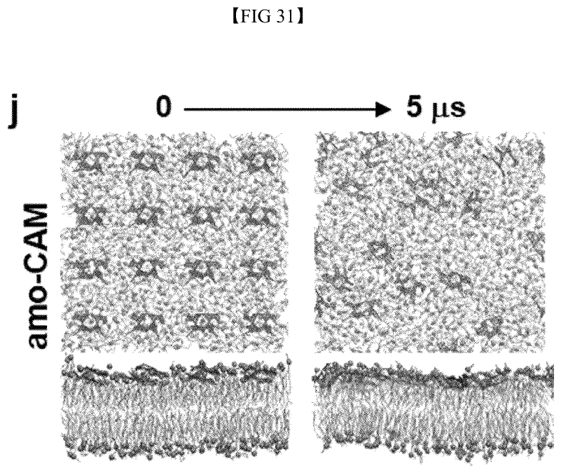

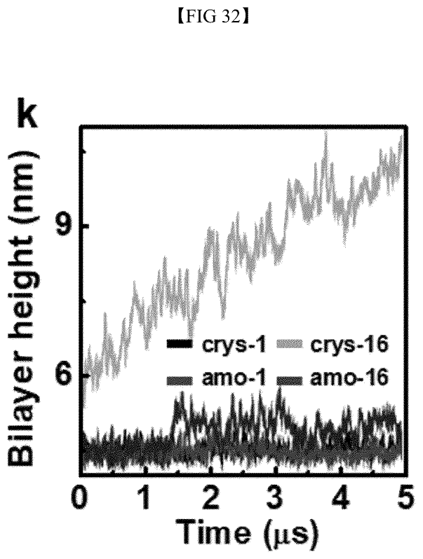

FIG. 31 shows height changes of membrane bilayers adsorbing crys-CAM (1 and 16 ea.) or amo-CAM (1 and 16 ea.).

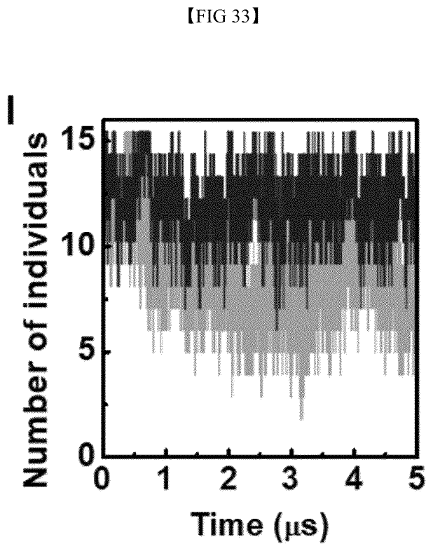

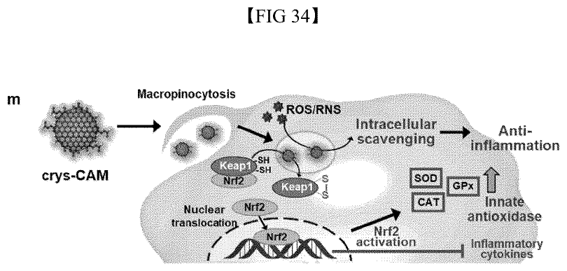

FIG. 33 shows numbers of crys-CAM or amo-CAM entities within non-clustered membrane bilayers over time, and FIG. 34 illustrates mechanisms of enhanced anti-inflammatory responses and innate immunity induced by SOD-like crys-CAM.

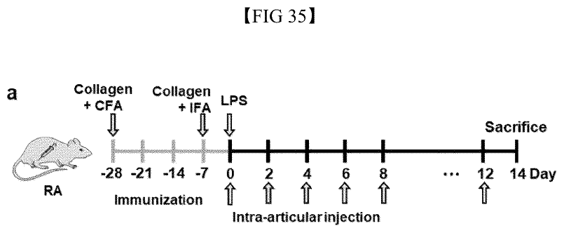

FIG. 35 shows an overall experimental schedule for rheumatoid arthritis (RA) mouse modeling and intra-articular injection of crys-CAM or amo-CAM.

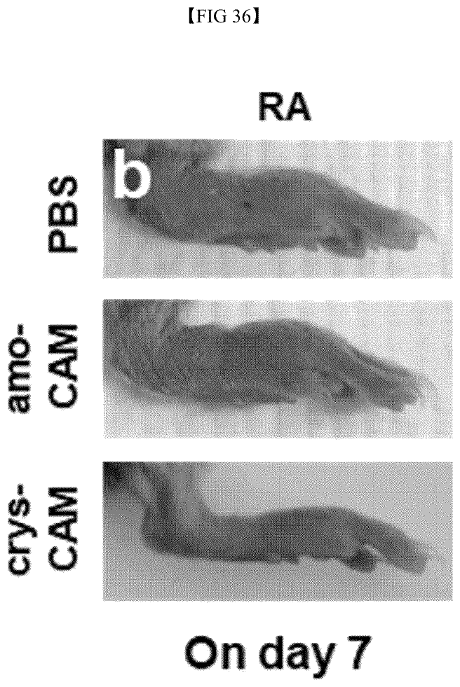

FIG. 36 shows optical images of mouse joints treated with PBS, amo-CAM, or crys-CAM on day7.

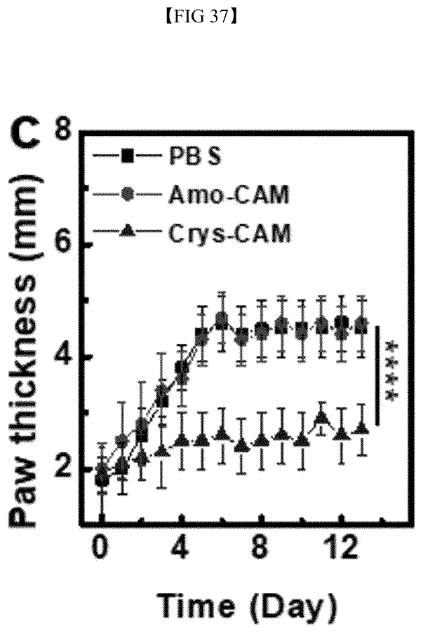

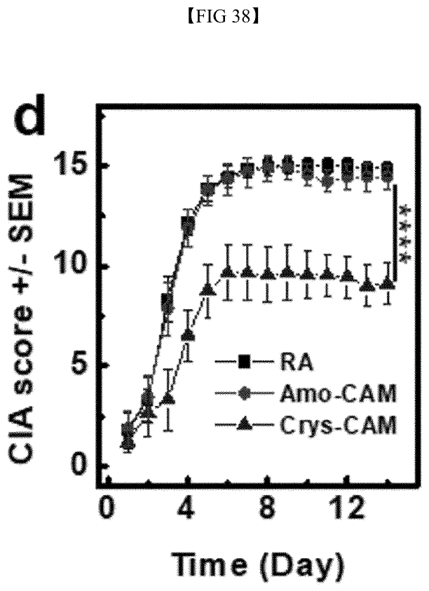

FIG. 37 shows paw thickness over time, and FIG. 38 shows the clinical CIA scores calculated for the joints of RA mice treated with PBS, amo-CAM, or crys-CAM over time.

Referring to FIGS. 36 through 38, it is confirmed that the crystalline mimetic exhibits sustained superior therapeutic efficacy according to the elapsed time after treatment.

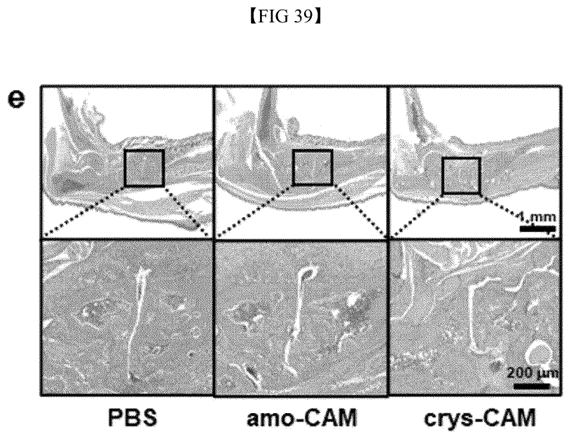

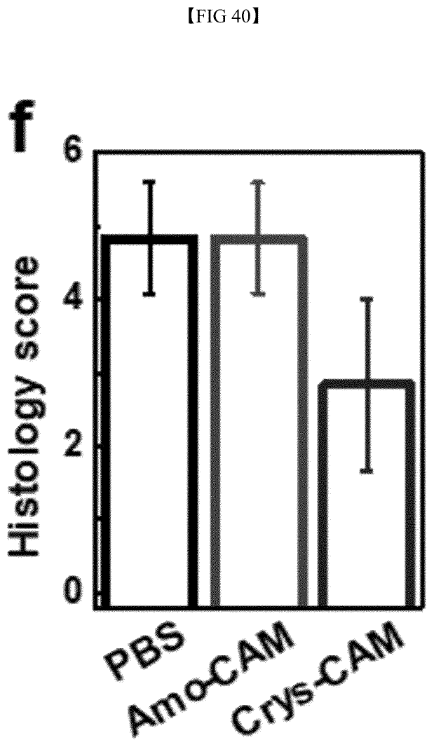

FIG. 39 shows Hematoxylin and Eosin (H&E)-stained joint images of RA mice treated with PBS, amo-CAM, or crys-CAM on day 4, and FIG. 40 illustrates histological scores for the corresponding H&E staining images.

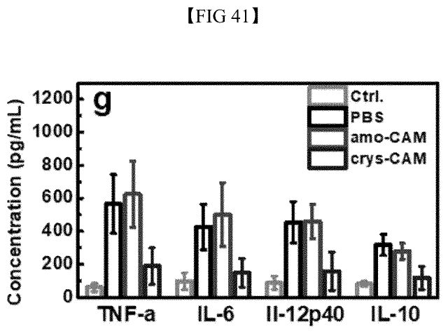

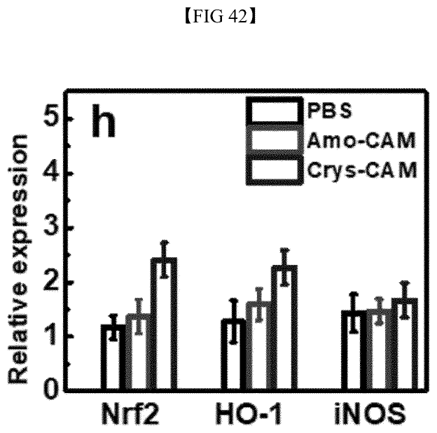

FIGS. 41 and 42 respectively illustrate the secretion levels of TNF-α, IL-6, IL-12p40, and IL-10 in synovial fluid of RA mice treated with PBS, amo-CAM, or crys-CAM at day 14, and the relative mRNA expression levels of Nrf2, HO-1, and iNOS.

### BEST MODE FOR CARRYING OUT THE INVENTION

Hereinafter, preferred embodiments of the present invention will be described in detail with reference to the accompanying drawings.

Before describing the present invention in detail, it should be understood that the terms or words used in the present specification are not to be construed as being limited to their ordinary or dictionary meanings. Rather, the inventor may appropriately define the concepts of such terms in order to explain the invention in the best manner. Accordingly, such terms and words should be interpreted as having meanings and concepts consistent with the technical spirit of the present invention.

The terms used herein are for the purpose of describing preferred embodiments only and are not intended to limit the scope of the present invention. These terms are defined in consideration of various possible embodiments of the present invention.

Unless otherwise clearly indicated by context, singular expressions include plural expressions, and plural expressions likewise include singular expressions.

Throughout the specification, when an element is described as “comprising” another element, it does not exclude the presence of additional elements unless expressly stated otherwise.

Further, when an element is described as being “disposed in” or “connected to” another element, the element may be directly connected to or in contact with the other element, or indirectly connected thereto.

To solve the above-described problems, the present invention provides a carbon-based antioxidant material functioning as an antioxidant enzyme mimetic, which effectively scavenges intracellular reactive oxygen species and reactive nitrogen species by controlling crystallinity of carbon nanoparticles.

In the present invention, the carbon antioxidant enzyme mimetic refers to a material having a carbon-based backbone and exhibiting antioxidant enzyme-like properties similar to superoxide dismutase (SOD).

The carbon antioxidant enzyme mimetic according to the present invention exhibits excellent antioxidant effects without using metal ions, through surface functional groups of a structure obtained by condensation polymerization of organic compounds.

For this purpose, the surface of the mimetic according to the present invention includes a first functional group selected from a carbonyl group or an amine group and a second functional group comprising a hydroxyl group. The mimetic is obtained by polymerizing caffeic acid (hydroxyl group, Example 1) or norepinephrine (Example 2).

Hereinafter, the present invention will be described in more detail through preferred examples and experimental examples. However, the scope of the present invention is not limited by these examples.

### MODE(S) FOR CARRYING OUT THE INVENTION

### Example 1

### Synthesis of Carbon Antioxidase Mimics (CAM)

Caffeic acid (81 mg), which is a low-molecular-weight organic compound having functional groups capable of undergoing condensation reactions, was dissolved in 15 mL of deionized water, and 200 μL of 1 M NaOH was added thereto. The resulting mixture was stirred while maintaining isothermal conditions by microwave irradiation at 150° C. for 10 minutes, thereby obtaining an amorphous carbon antioxidase mimics (amo-CAM).

For synthesis of a crystalline carbon antioxidase mimics (crys-CAM), the same reaction was performed at 210° C. for 20 minutes.

After completion of the reaction, the solution was dialyzed against 5 μL of water using a cellulose membrane for 3 hours, and this dialysis process was repeated five times, thereby obtaining crystalline and amorphous carbon-based antioxidant mimetics. Thereafter, the crys-CAM or amo-CAM solution was filtered through a polyvinylidene fluoride (PVDF) filter having a pore size of 100 nm, and the filtrate containing the target product was obtained.

FIG. 1 schematically illustrates the synthesis of crystalline or amorphous carbon antioxidase mimics (crys-CAM or amo-CAM) through a spontaneous solvothermal reaction of caffeic acid. As shown in FIG. 1, crystallinity of the carbon antioxidant enzyme mimetic varies depending on microwave irradiation time and temperature.

The antioxidant enzyme mimetic according to one embodiment of the present invention has carbonyl, hydroxyl, and carboxyl groups on its surface. In particular, carbonyl and hydroxyl groups formed on the surface of the crystalline mimetic simultaneously bind to reactive oxygen species (HOO⁻) and oxidize the same to generate O₂. Subsequently, two hydroxyl groups bind again to HOO⁻ to generate H₂O₂.

The reaction can be summarized as follows:

2HOO⁻→O₂+H₂O₂

Accordingly, the mimetic according to the present invention can selectively oxidize or reduce HOO⁻, which is attributable to its structural characteristics of simultaneously having carbonyl and hydroxyl groups.

### Example 2

### Synthesis Using Norepinephrine

A carbon-based structure was synthesized in the same manner as in Example 1, except that norepinephrine represented by the following chemical formula was used instead of caffeic acid. In this case, it can be confirmed that an amine group was introduced instead of a carboxyl group.

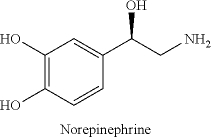 [SMILES: NC[C@H](O)c1ccc(O)c(O)c1]

### Experimental Example 1

### Measurement of ROS/RNS Scavenging Activity

In order to measure the superoxide scavenging activity of Examples or Comparative Examples, superoxide anions were generated by xanthine (Xan)/xanthine oxidase (XOD) reactions. To this end, a reaction mixture was prepared by mixing 50 μL of Xan (1 mM), 125 μL of crys-CAM (0 to 200 μM), and 15 μL of WST-8, followed by addition of 50 μL of XOD (0.1 U/mL). The mixture was shaken at 25° C. for 2 hours, and absorbance at 460 nm was measured using a microplate reader.

To assess scavenging activity against hydroxyl radicals (⋅OH), 6 mM H₂O₂, 0.1 mg/mL of TMB, 20 μL of FeSO₄ (400 μM), and 200 μL of crys-CAM (0 to 200 μM) were mixed and stirred at 25° C. for 1 hour. The reaction was then stopped by adding 15 μL of 2 M H₂SO₄ to the mixture, and absorbance at 450 nm was measured using a microplate reader.

For nitric oxide (NO) scavenging activity of Examples or Comparative Examples, NO was generated using sodium nitroprusside (SNP). First, 125 μL of SNP (20 mM) and 125 μL of crys-CAM (0 to 200 μM) were mixed with a phosphate-buffered saline (PBS) solution (0.1 M, pH 7.4) and stirred at 25° C. for 2 hours. After addition of 7.5 μL of nitrate reductase and 7.5 L of enzyme cofactor to the crys-CAM solution, the mixture was further stirred at 25° C. for 1 hour. Subsequently, 50 μL of Griess reagent R1 and 50 μL of Griess reagent R2 were added to the crys-CAM solution, and the mixture was stirred at 25° C. for 30 minutes. Absorbance of the solution was then measured at 540 nm using a microplate reader.

### Measurement of Intracellular ROS and RNS

Intracellular reactive oxygen species (ROS) and reactive nitrogen species (RNS) levels were assessed using intracellular fluorescence-based assays.

For this purpose, cells were cultured in serum-free medium and loaded with redox-sensitive fluorescent dyes as follows: 10 μM 2′,7′-dichlorofluorescein diacetate (DCFH-DA; Calbiochem) for detection of H₂O₂; OH580 probe (hydroxyl radical detection assay kit, ab219931; Abbacam) for detection of hydroxyl radicals; 2 μM dihydroethidium (DHE; Calbiochem) for detection of superoxide (02); or 10 μM 4,5-diaminofluorescein diacetate (DAF-2DA; Calbiochem) for detection of nitric oxide (NO).

After dye loading, cells were washed by pulse spinning and immediately analyzed using a FACSCalibur flow cytometer (BD Biosciences, San Jose, CA). Data acquisition, analysis, and plotting were performed using CellQuest software (BD Biosciences).

### Measurement of Natural Antioxidant Enzyme Activity Inside Cells

Total protein levels and activities of endogenous antioxidant enzymes, including superoxide dismutase (SOD), glutathione peroxidase (GPx), catalase (CAT), and glutathione reductase (GSR), were evaluated using established methods.

Activities of SOD, GPx, and GSR were measured using Randox assay kits (Randox Laboratories Ltd., Crumlin, UK) adapted for use with an Olympus AU400 system. Specifically, GPx activity (Ransel kit) was determined by monitoring oxidation of NADPH in the presence of cumene hydroperoxide. GSR activity (Glut Red kit) was measured by monitoring NADPH oxidation, and SOD activity (Ransod kit) was evaluated using a xanthine oxidase (XOD)-based method. CAT activity was determined indirectly by monitoring consumption of H₂O₂ according to established methods.

### Experimental Results

In the present experiment, it was confirmed that the carbon-based antioxidant enzyme mimetic according to an embodiment of the present invention exhibits a high antioxidant effect by effectively scavenging ABTS radicals both intracellularly and extracellularly. These results outperform Trolox, a representative organic antioxidant molecule, and are described in more detail with reference to the accompanying drawings.

FIG. 2 is a TEM image of a crystalline or amorphous carbon-based antioxidant enzyme mimetic prepared according to the present invention, wherein the upper image corresponds to the amorphous structure and the lower image corresponds to the crystalline structure.

Referring to FIG. 2, it can be seen that the carbon-based antioxidant enzyme mimetic according to the present invention has an average particle size of approximately 5.2 nm.

FIG. 3 shows FFT patterns of a crystalline or amorphous carbon-based antioxidant enzyme mimetic prepared according to the present invention, wherein the upper pattern corresponds to the amorphous structure and the lower pattern corresponds to the crystalline structure.

Referring to FIG. 3, it can be observed that a lattice spacing corresponding to 0.23 nm appears in the crystalline carbon-based antioxidant enzyme mimetic.

FIG. 4 is an AFM image of a crystalline or amorphous carbon-based antioxidant enzyme mimetic prepared according to the present invention, wherein the upper image corresponds to the amorphous structure and the lower image corresponds to the crystalline structure.

Referring to FIG. 4, it can be seen that the mimetic according to the present invention has a thickness of approximately 1.9 nm.

FIG. 5 shows UV-Vis absorption spectra of a crystalline or amorphous carbon-based antioxidant enzyme mimetic prepared according to the present invention.

Referring to FIG. 5, it can be confirmed that absorbance of the crystalline carbon-based antioxidant enzyme mimetic is higher at 450 nm, indicating higher crystallinity.

FIG. 6 shows Raman spectra of a crystalline or amorphous carbon-based antioxidant enzyme mimetic prepared according to the present invention.

Referring to FIG. 6, it can be confirmed that the crystalline carbon-based antioxidant enzyme mimetic exhibits higher crystallinity, as evidenced by a lower D/G peak intensity ratio.

FIG. 7 shows XPS spectra of a crystalline or amorphous carbon-based antioxidant enzyme mimetic prepared according to the present invention, wherein the left graph (k) corresponds to the amorphous structure and the right graph (i) corresponds to the crystalline structure.

Referring to FIG. 7, it can be seen that the antioxidant enzyme mimetic according to the present invention includes carboxyl groups, carbonyl groups, and hydroxyl groups regardless of crystallinity.

FIG. 8 shows analysis results of the antioxidant mechanism and scavenging activity of crys-CAM, amo-CAM, and Trolox against ABTS radicals according to Example 1 of the present invention.

FIG. 9 shows concentrations required to scavenge 50% of ABTS radicals (SC₅₀) and scavenging rate constants of crys-CAM and amo-CAM according to Example 1 of the present invention for extracellular peroxide.

Referring to the results of FIGS. 8 and 9, both crystalline and amorphous antioxidant enzyme mimetics according to the present invention exhibit significantly stronger antioxidant effects than Trolox, a well-known organic antioxidant, and also demonstrate substantially faster antioxidant reaction rates.

FIG. 10 shows results of concentration-dependent scavenging activity analysis of crys-CAM and amo-CAM according to Example 1 with respect to extracellular superoxide.

FIG. 11 shows a Lineweaver-Burk plot for elimination of excess superoxide by SOD-like crys-CAM according to Example 1.

FIG. 12 shows results of analyzing the amount of molecular oxygen generated from superoxide by SOD-like crys-CAM according to Example 1.

FIG. 13 shows results of analyzing scavenging activity of crys-CAM and amo-CAM according to Example 1 with respect to extracellular nitric oxide, and FIG. 14 shows results of analyzing scavenging activity with respect to hydroxyl radicals.

FIG. 15 is a schematic diagram illustrating treatment of crys-CAM according to Example 1 with NaBH₄ and 1,3-propane sultone (PS), and FIG. 16 shows results of analyzing SOD-like activity of untreated crys-CAM and crys-CAM treated with NaBH₄ and PS.

FIG. 17 is a diagram illustrating the mechanism of SOD-like activity of crys-CAM according to Example 1.

Referring to FIG. 17, the antioxidant enzyme mimetic according to an embodiment of the present invention includes carbonyl groups, hydroxyl groups, and carboxyl groups on its surface. In particular, carbonyl groups and hydroxyl groups formed on the surface of the crystalline mimetic simultaneously bind to reactive oxygen species (HOO⁻) and oxidize the same to generate O₂. Thereafter, two hydroxyl groups bind again to HOO⁻ to generate H₂O₂.

The reaction is summarized as follows:

2HOO⁻→O₂+H₂O₂

FIG. 18 shows results of comparing scavenging activity against intracellular superoxide, nitric oxide, hydroxyl radicals, and hydrogen peroxide in cells treated with amo-CAM (a) and crys-CAM (b).

Referring to FIG. 18, it can be seen that the crystalline mimetic according to the present invention is efficiently internalized into cells and exhibits superior intracellular antioxidant effects.

FIG. 19 illustrates antioxidant properties of a mimetic synthesized according to Example 2 and comprising an amine group.

Referring to FIG. 19, it can be seen that the mimetic of Example 2 containing an amine group exhibits antioxidant properties comparable to those of the mimetic of Example 1.

### Experimental Example 2

### Molecular Kinetics Simulation of Cell Permeability of Carbon Antioxidase Mimics (CAM)

All simulations and analyses were performed using the GROMACS 2018.6 simulation package with the MARTINI 2.2 coarse-grained (CG) force field, in which several (three or four) heavy atoms are grouped into a single CG bead.

For the carbon antioxidase mimics (CAM) according to the present invention, hydrocarbons in aromatic rings, hydroxyl groups (—OH), and anionic carbonyl groups (C═O) were modeled as bead types of hydrophobic “SC2,” polar “SPI,” and negatively charged “SQa,” respectively.

The crystalline crys-CAM consisted of 51 SC2, 10 SPI, and 4 SQa beads, whereas the amorphous amo-CAM consisted of 29 SC2, 20 SPI, and 2 SQa beads, consistent with the CAM structures characterized experimentally.

To mimic membranes of human macrophages, the phospholipid bilayer model consisted of 1-palmitoyl-2-oleoyl-sn-glycero-3-phosphocholine (POPC; 28 mol %), 1-palmitoyl-2-oleoyl-sn-glycero-3-phosphoethanolamine (POPE; 10 mol %), 1-palmitoyl-2-oleoyl-sn-glycero-3-phosphoglycerol (POPG; 8 mol %), 1-palmitoyl-2-oleoyl-sn-glycero-3-phosphoinositol (POPI; 5.5 mol %), 1-palmitoyl-2-oleoyl-sn-glycero-3-phospho-L-serine (POPS; 3.5 mol %), sphingomyelin (SM(d18:1/18:0); 10.5 mol %), and cholesterol (CHOL; 34.5 mol %), corresponding to experimentally reported lipid compositions. Considering the presence of anionic lipids on the inner leaflet of human cell membranes, POPG, POPS, and POPI lipids were located on the inner leaflet, whereas the outer leaflet consisted predominantly of CHOL, POPC, and SM lipids.

A single CAM molecule or 16 CAM molecules were initially placed on the outer surface of an equilibrated bilayer, which was solvated with approximately 8,100 CG water beads (corresponding to 32,400 real water molecules) in a periodic simulation box of 14×14×9 nm³. Electrical neutrality was achieved by adding sufficient counterions (Na⁺), and an additional 100 Na⁺/Cl⁻ ion pairs were added to produce a salt concentration of 0.14 M NaCl, close to experimental conditions.

A cutoff of 1.1 nm was used for the Lennard-Jones potential with a smooth transition to zero between 0 and 1.1 nm. For Coulombic interactions, a cutoff of 1.1 nm with a relative dielectric constant of 15 was applied. The system temperature was maintained at 310 K and the pressure at 1 bar using the velocity-rescale thermostat and the Parrinello-Rahman barostat under an NPxyPzT ensemble with anisotropic pressure coupling. Bond lengths were constrained using the LINCS algorithm.

The simulations were performed for 5 s with a time step of 20 fs at a computing facility supported by the National Supercomputing Center using supercomputing resources (KSC-2023-CRE-019), including technical support. The final 2 s of each trajectory were averaged for analysis.

### Collagen-Induced Arthritis Mouse Model

Bovine type II collagen (Chondrex Inc., WA, USA) was dissolved at a concentration of 2 mg/mL and emulsified with complete Freund's adjuvant (CFA; Chondrex Inc.) containing heat-inactivated Mycobacterium tuberculosis. Mice were immunized by intradermal injection of 100 μL of the collagen/CFA emulsion.

Twenty-one days after the first immunization, the mice received a second intradermal injection of 100 μL of an emulsion of type II collagen and incomplete Freund's adjuvant (IFA; Chondrex Inc.). Subsequently, one week after the second immunization, 50 μL of a partial LPS solution (1 mg/mL in PBS, pH 7.4) was injected intraperitoneally to enhance the inflammatory response, as previously described. Simultaneously, collagen-induced arthritis (CIA) mice received intra-articular injections of crys-CAM (16 mg/kg), amo-CAM (16 mg/kg), or PBS.

### Arthritis Evaluation

One week after the second immunization, trained investigators conducted a blinded assessment of arthritis severity. Arthritis severity was evaluated daily, and each paw was scored based on the degree of redness and swelling. Each paw was assigned a score ranging from 0 (no symptoms) to a maximum of 4.

For histological analysis, mice were euthanized, and hind paws were harvested, fixed in 10% buffered formalin, decalcified using 10% (w/v) EDTA, and embedded in paraffin. Tissue sections were prepared and subjected to hematoxylin and eosin (H&E) staining for histological examination.

### Experimental Example 3

In the present experimental example, the therapeutic effect of the carbon-based antioxidant enzyme-like matrix according to an embodiment of the present invention on rheumatoid arthritis was evaluated. As a result, in contrast to the amorphous carbon antioxidase mimics (amo-CAM), the crystalline carbon antioxidase mimics (crys-CAM) exhibited superior endocytosis. Following intracellular uptake, reactive oxygen species and reactive nitrogen species were effectively eliminated, thereby producing a therapeutic effect against rheumatoid arthritis. These results are described in further detail with reference to the accompanying drawings.

FIG. 20 shows secretion levels of TNF-α (acute inflammation), IL-6 (chronic inflammation), IL-12p40 (chronic inflammation), and IL-10 (anti-inflammatory) in cells treated with amo-CAM (a) or crys-CAM (b).

Referring to FIG. 20, it may be seen that levels of pro-inflammatory cytokines are significantly reduced upon treatment with crystalline crys-CAM.

FIG. 21 shows results of Western blot analysis of the Nrf2 signaling pathway in LPS-stimulated bone marrow-derived macrophage (BMDM) after treatment with amo-CAM or crys-CAM.

Referring to FIG. 21, it may be seen that activation of the Nrf2 signaling pathway occurs selectively in cells treated with the crystalline carbon-based antioxidant enzyme-like matrix, which is consistent with the results shown in FIG. 9.

FIG. 22 shows intracellular activities of Nrf2-associated innate antioxidant enzymes, including SOD, CAT, and GPx, in LPS-stimulated BMDM.

Referring to FIG. 22, it may be seen that only the crystalline carbon-based antioxidant enzyme-like matrix according to the present invention increases intracellular antioxidant enzyme activity through activation of the Nrf2 signaling pathway.

FIGS. 23 to 26 show Western blot analyses evaluating the effects of amo-CAM and crys-CAM on mitochondrial ROS-producing proteins in LPS-stimulated BMDM under various concentration conditions, as well as activities of Complex I, Complex III, and NADPH oxidase.

Referring to FIGS. 23 to 26, it may be seen that the carbon-based antioxidant enzyme-like matrix according to the present invention does not directly interfere with endogenous ROS-generating mechanisms.

FIG. 27 shows optical and fluorescence images of RAW264.7 cells treated with PBS, amo-CAM, or crys-CAM, illustrating intracellular uptake pathways and enhancement of anti-inflammatory immunity.

FIG. 28 quantitatively illustrates fluorescence intensity corresponding to FIG. 27.

Referring to FIGS. 27 and 28, it may be seen that only the crystalline carbon-based antioxidant enzyme-like matrix according to the present invention is efficiently internalized by cells.

FIG. 29 shows effects of endocytosis inhibitors on cellular uptake of crys-CAM in RAW264.7 cells.

Referring to FIG. 29, it may be seen that crys-CAM is internalized predominantly through macropinocytosis.

FIG. 30 shows snapshots of top and side views of cell membranes adsorbing crys-CAM (red beads) or amo-CAM (blue beads) over time.

FIG. 31 shows changes in membrane bilayer height upon adsorption of crys-CAM (1 or 16 entities) or amo-CAM (1 or 16 entities).

Referring to FIGS. 30 and 31, it may be seen that the crystalline carbon-based antioxidant enzyme-like matrix forms clusters on the cell surface and exhibits strong interactions with the cell membrane, facilitating cellular internalization.

FIG. 33 shows the number of crys-CAM or amo-CAM entities within non-clustered membrane bilayers overtime, and FIG. 34 schematically illustrates mechanisms underlying enhanced anti-inflammatory and innate immune responses induced by SOD-like crys-CAM.

FIG. 35 illustrates an overall experimental schedule for rheumatoid arthritis (RA) mouse modeling and intra-articular injection of crys-CAM or amo-CAM.

FIG. 36 shows optical images of mouse joints treated with PBS, amo-CAM, or crys-CAM on day 1, FIG. 37 shows changes in paw thickness over time, and FIG. 38 shows clinical CIA scores of RA mice treated with PBS, amo-CAM, or crys-CAM.

Referring to FIGS. 36 to 38, it may be seen that the crystalline carbon-based antioxidant enzyme-like matrix exhibits sustained therapeutic efficacy throughout the treatment period.

FIG. 39 shows H&E-stained joint sections of RA mice treated with PBS, amo-CAM, or crys-CAM on day 4, and FIG. 40 shows corresponding histological scores.

FIGS. 41 and 42 show secretion levels of TNF-α, IL-6, IL-12p40, and IL-10 in synovial fluid of RA mice treated with PBS, amo-CAM, or crys-CAM on day 14, as well as relative mRNA expression levels of Nrf2, HO-1, and iNOS.

### INDUSTRIAL APPLICABILITY

The present invention relates to a carbon antioxidant enzyme mimetic, an anti-inflammatory therapeutic composition comprising the same, and a method for preparing the same, and therefore has industrial applicability.

What is claimed is:

1. A carbon antioxidant enzyme mimetic,
wherein a surface of the carbon antioxidant enzyme mimetic comprises:
a first functional group selected from a carbonyl group or an amine group; and
a second functional group comprising a hydroxyl group.

2. The carbon antioxidant enzyme mimetic according to claim 1,
wherein the first functional group and the second functional group simultaneously participate in an oxidation reaction of a reactive oxygen species HOO⁻.

3. The carbon antioxidant enzyme mimetic according to claim 2,
wherein the carbon antioxidant enzyme mimetic selectively oxidizes or reduces the reactive oxygen species HOO⁻.

4. The carbon antioxidant enzyme mimetic according to claim 2,
wherein, after the oxidation reaction, the first functional group comprising the carbonyl group is converted into the hydroxyl group, and the second functional group comprising the hydroxyl group subsequently binds to the reactive oxygen species HOO⁻ to reduce the same.

5. The carbon antioxidant enzyme mimetic according to claim 4,
wherein the carbon antioxidant enzyme mimetic removes the reactive oxygen species HOO⁻ according to the following reaction scheme:
2HOO⁻→O₂+H₂O₂.

6. The carbon antioxidant enzyme mimetic according to claim 1,
wherein the carbon antioxidant enzyme mimetic has crystallinity, and a crystalline domain size of the carbon antioxidant enzyme mimetic is 0.2 nm or greater.

7. An anti-inflammatory therapeutic composition comprising, as an active ingredient, a carbon antioxidant enzyme mimetic according to claim 1 or a pharmaceutically acceptable salt thereof,
wherein the carbon antioxidant enzyme mimetic is crystalline.

8. The anti-inflammatory therapeutic composition according to claim 7,
wherein intracellular uptake of the carbon antioxidant enzyme mimetic is induced by crystallinity.

9. The anti-inflammatory therapeutic composition according to claim 8,
wherein the anti-inflammatory therapeutic composition is for treating rheumatoid arthritis.
